(19) United States
(12) Patent Application Publication
(10) Pub. No.: US 2014/0128812 A1
(43) Pub. Date: May 8, 2014

(54) T-SHAPED GASTROSTOMY TUBE RETENTION DEVICE

(71) Applicant: COOK MEDICAL TECHNOLOGIES LLC, Bloomington, IN (US)

(72) Inventor: Kevin Delaney, Clifton Park, NY (US)

(73) Assignee: Cook Medical Technologies LLC, Bloomington, IN (US)

(21) Appl. No.: 13/668,907

(22) Filed: Nov. 5, 2012

(51) Int. Cl. A61M 39/00

(52) U.S. Cl. 604/174

(57) ABSTRACT

A tube retention device may prohibit the removal of a tube from a body cavity. The tube retention device may include an elongate segment connected to the tube through a rotatable hinge. The elongate segment may have a length larger than the diameter of the tube. The elongate segment may rotate about the rotatable hinge from an insertion position, in which the length of the elongate segment is parallel to the tube, to a retention position, in which the length of the elongate segment is perpendicular to the tube. In the retention position, engagement of the elongate segment with the interior surface of the body cavity may prohibit removal of the tube from the body cavity.

### BACKGROUND OF THE INVENTION

1. Field of the Invention

The invention relates to medical devices, more particularly to the retention of tubes inside a body cavity.

2. Description of the Related Art

Tubes are inserted into bodies for multiple reasons, such as feeding tubes or drainage tubes. Feeding tubes may be required to provide nutrition if normal ingestion of food becomes difficult or impossible for a patient. Certain patients, such as comatose patients, stroke victims, or those with a compromised gastrointestinal tract, may require a tube that is introduced to the stomach or intestinal tract through a surgical opening in the body, such as a stoma. Once inserted into the body cavity, an internal retention device is required to ensure the tube stays in the desired location and is not inadvertently removed by the patient.

Various types of retention devices exist, including balloons, deformable collars, and permanently attached flanges. However, none of the existing retention devices securely retain the tube within the body cavity, require little or no maintenance, and also minimize the size of the surgical opening required to deliver the tube and retention device. Balloons require inflation once inside the body cavity and require periodic maintenance to verify the balloon's volume remains adequate to securely retain the tube. Furthermore, the relatively large size of the balloon can block internal body passageways or otherwise interfere with bodily functions. Deformable collars include complex and expensive elements that deform based on force applied from the exterior of the body. Permanently attached flanges include deformable retaining elements that allow the device to collapse when force is applied, however, the deformability also reduces the resistance to removing the device.

Thus, there is a need for a tube retention device that securely holds the tube inside the body cavity without the problems of the prior art devices.

### SUMMARY OF THE INVENTION

The descriptions below include devices for retaining tubes within body cavities. The tube retention devices may include elongate segments that rotate about a rotatable hinge to engage the interior surface of the body cavity after insertion into the body cavity. During insertion, the elongate segments may be rotated to a position parallel with the tube such that the surgical opening in the body is minimized.

According to one embodiment of the invention, a device for retaining a tube inside a body cavity comprises an elongate segment attached to the distal end of a tube, where a length of the elongate segment is larger than the diameter of the tube and where the length of the elongate segment is operable to be substantially parallel to the tube in an insertion position and the length of the elongate segment is operable to be substantially perpendicular to the tube in a retention position, and a rotatable hinge connecting the elongate segment to the distal end of the tube.

According to another embodiment of the invention, a catheter comprises a tube with a distal end and a proximal end, where the tube comprises a lumen connecting the distal end and the proximal end, and a retention device located at the distal end of the tube, where the retention device includes an elongate segment, where the length of the elongate segment is larger than the diameter of the tube and where the length of the elongate segment is operable to be parallel to the tube in an insertion position and the length of the elongate segment is operable to be perpendicular to the tube in a retention position, and a rotatable hinge connecting the elongate segment to the distal end of the tube.

According to another embodiment of the invention, a method of retaining a tube inside a body cavity comprises inserting a distal end of a tube inside a body cavity, where a rotatable hinge connects an elongate segment to the distal end of the tube, and rotating the elongate segment about the rotatable hinge to a position that is perpendicular to the tube, where a length of the elongate segment is larger than the diameter of the tube.

Other systems, methods, features and advantages will be, or will become, apparent to one with skill in the art upon examination of the following figures and detailed description. It is intended that all such additional systems, methods, features and advantages be included within this description, be within the scope of the invention, and be protected by the following claims.

### BRIEF DESCRIPTION OF THE DRAWINGS

FIG. 1A is an elevation view of a tube retention device in an insertion position according to one embodiment of the invention;

FIG. 1B is a side view of the tube retention device of FIG. 1A in an insertion position;

FIG. 2A is an elevation view of the tube retention device of FIG. 1A in a retention position;

FIG. 2B is a side view of the tube retention device of FIG. 2A in a retention position;

FIG. 2C is a top view of the tube retention device of FIG. 2A in a retention position;

FIG. 3A is an elevation view of a tube retention device in an insertion position according to another embodiment of the invention;

FIG. 3B is a side view of the tube retention device of FIG. 3A in an insertion position;

FIG. 4A is an elevation view of the tube retention device of FIG. 3A in a retention position;

FIG. 4B is a side view of the tube retention device of FIG. 4A in a retention position;

FIG. 4C is a top view of the tube retention device of FIG. 4A in a retention position;

FIG. 5A is an elevation view of a tube retention device in an insertion position according to another embodiment of the invention;

FIG. 5B is a side view of the tube retention device of FIG. 5A in an insertion position;

FIG. 6A is an elevation view of the tube retention device of FIG. 5A in a retention position;

FIG. 6B is a side view of the tube retention device of FIG. 6A in a retention position;

FIG. 6C is a top view of the tube retention device of FIG. 6A in a retention position;

FIG. 7 is a flow diagram of a method for retaining a tube inside a body cavity according to another embodiment of the invention.

### DETAILED DESCRIPTION OF THE PREFERRED EMBODIMENTS

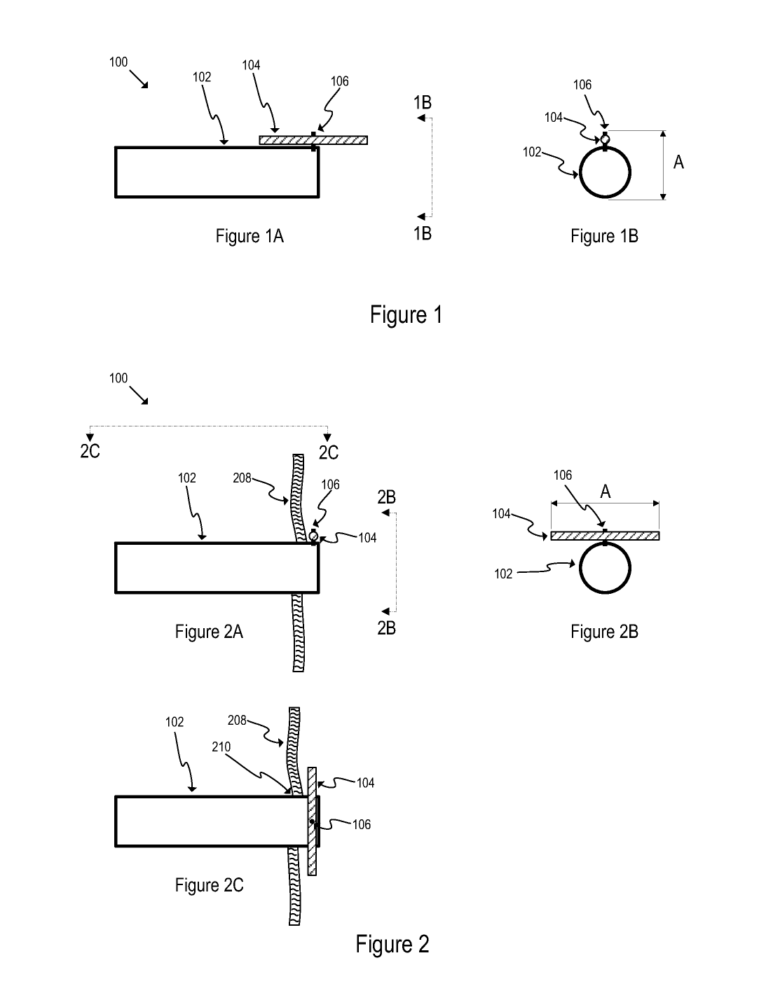

Various embodiments of the tube retention device 100 according to the present invention are shown in FIGS. 1-7. In general, the tube retention device 100 may comprise an elongate segment 104 attached to the distal end of a tube 102 through a rotatable hinge 106. Rotatable hinge 106 may allow elongate segment 104 to spin or rotate around the connection point such that the position of elongate segment 104 in reference to tube 102 may be modified. Elongate segment 104 may have at least one dimension that is larger than the diameter of tube 102. As will be explained in greater detail below, during insertion into the body cavity, elongate segment 104 may be positioned substantially parallel to tube 102. Parallel position of elongate segment 104 may allow the diameter of the surgical opening from the exterior of the body to the interior of the body to be minimized. The opening may be substantially the same size as the diameter of the tube to be inserted into the opening. After insertion into the body cavity, elongate segment 104 may be rotated about rotatable hinge 106 such that elongate segment 104 is positioned substantially perpendicular to tube 102. Perpendicular position of elongate segment 104 may cause tube retention device 100 to be oriented such that removal of tube 102 is prevented. Removal is prevented because the dimension of elongate segment 104 adjacent to the opening is larger than the diameter of the opening.

A smaller diameter tube may be inserted into the lumen of tube 102. The smaller diameter tube may be used to deliver products from the exterior of the body into the body cavity. Alternatively, tube 102 may be used to deliver products from the exterior of the body into the body cavity. A smaller diameter tube inserted through the lumen of tube 102, or tube 102 itself, may also be used to extract fluids or objects from the body cavity.

Prior to removal of tube 102, elongate segment 104 may be rotated back to a position substantially parallel to tube 102 such that the dimension of elongate segment 104 adjacent to the surgical opening is substantially the same size as the diameter of the opening. Tube 102 and tube retention device 100 may then be easily removed from the body without enlarging the original surgical opening.

In the embodiment in FIG. 1, tube retention device 100 may comprise an elongate segment 104 attached to the distal end of a tube 102 through a rotatable hinge 106. A proximal end of tube 102 may be designed to remain outside of a body, while the distal end of tube 102 may be designed to enter into and remain inside the body. Tube retention device 100 may be designed for any open cavity inside a body, such as, for example, stomach, other portion of the gastrointestinal system, bladder, or portion of the respiratory system. Tube retention device 100 may be used with any type of indwelling tube, such as, for example a feeding tube, catheter, or other element with a lumen. Tube retention device 100 may be located anywhere that is proximal to the distal end of tube 102 and that is designed to be placed inside the body cavity.

Elongate segment 104 may be configured to engage the interior surface of the body cavity, such as a stomach wall, after tube retention device 100 is placed inside the body cavity. Elongate segment 104 may be any shape, such as, for example, cylindrical, oblong, oval, rectangular, triangular, hemispherical, or polygon. Elongate segment 104 may have at least one dimension that is larger than the diameter of tube 102. Preferably, other dimensions of elongate segment 104 are smaller than or equal to the diameter of tube 102. Elongate segment 104 may be comprised of material that is designed to be located within a body for an extended period of time, such as, for example, silicon, polyurethane, inert metal, or other biocompatible material. Elongate segment 104 may be comprised of the same material as tube 102 or may be comprised of a different material. Preferably, elongate segment 104 may be comprised of a resilient material that is more rigid than the material comprising tube 102. Elongate segment 104 may be partially or fully hollow or may be solid.

Rotatable hinge 106 connects elongate segment 104 to tube 102. Rotatable hinge 106 may be any type of joining method that attaches elongate segment 104 to tube 102 and allows elongate segment 104 to rotate or pivot about the connection point. Preferably, rotatable hinge 106 may allow elongate segment 104 to rotate at least ninety degrees relative to the connection point or tube 102. Rotatable hinge 106 may also allow elongate segment 104 to rotate three hundred and sixty degrees relative to the connection point or tube 102. Rotatable hinge 106 may be, for example, a tether, flexible wire, rivet, or other type of mechanical fastener. Rotatable hinge 106 may also be, for example, a barrel hinge, a spherical hinge, or a flag hinge. Rotatable hinge 106 may include a male component fitted between female components located on tube 102 and elongate segment 104. Alternatively, rotatable hinge 106 may include a male component attached to either tube 102 or elongate segment 104 and a corresponding female component located on tube 102 or elongate segment 104. Preferably, rotatable hinge 106 comprises a pin style hinge where one end of the pin is attached to tube 102 and the other end of the pin is attached to elongate segment 104. Rotatable hinge 106 may include burs, raised portions, or other means to prevent the tube or elongate segment 104 from becoming detached from rotatable hinge 106. Rotatable hinge 106 may provide some resistance to the rotation of elongate segment 104. Preferably, rotatable hinge 106 may allow elongate segment 104 to freely rotate about the connection point. Rotatable hinge 106 may be comprised of material that is configured to be located within a body for an extended period of time, such as, for example, silicon, polyurethane, inert metal, or other biocompatible material. Preferably, rotatable hinge 106 may be comprised of a material that is designed to not significantly degrade, permanently deform, or break throughout the expected design life of tube retention device 100. Rotatable hinge 106 may be partially or fully hollow or may be solid.

Rotatable hinge 106 may be connected to elongate segment 104 at any location on elongate segment 104 that allows elongate segment 104 to rotate about the connection point. Preferably, rotatable hinge 106 may be connected to elongate segment 104 near, such as plus or minus twenty percent, the midpoint of the longest dimension of elongate segment 104. Connecting the midpoint of elongate segment 104 to tube 102 provides a substantially balanced surface area on each side of rotatable hinge 106 to engage the interior surface of the body cavity. As such, connecting the midpoint of elongate segment 104 to tube 102 may provide the most effective resistance to removing tube 102 from within the body cavity after elongate segment 104 has been rotated to a retention position.

FIGS. 1A and 1B illustrate different viewpoints of tube retention device 100 in an insertion position according to an embodiment of the invention. FIG. 1A shows an elevation view of tube retention device 100 in an insertion position, while FIG. 1B shows a side view of tube retention device 100 in an insertion position. An insertion position may allow tube 102 and tube retention system 100 to enter the body cavity through the smallest possible surgical opening in the body because the largest dimension of the combined tube 102 and tube retention device 100 that is parallel to the body, shown as dimension A in FIG. 1B, is minimized. Tube retention device 100 may be in an insertion position when the largest dimension of elongate segment 104 is substantially parallel to tube 102. It may not be necessary for elongate segment 104 to be exactly parallel to tube 102 in an insertion position. To reach an insertion position, it may be sufficient for elongate segment 104 to be substantially parallel to tube 102, such that the rotational position of elongate segment 104 does not increase the largest dimension of the combined tube 102 and tube retention device 100 that is parallel to the body.

FIGS. 2A, 2B, and 2C illustrate different viewpoints of tube retention device 100 in a retention position. FIG. 2A shows an elevation view of tube retention device 100 in a retention position, FIG. 2B shows a side view of tube retention device 100 in a retention position, and FIG. 2C shows a top view of tube retention device 100 in a retention position. The wall of the body cavity 208 is shown in FIGS. 2A and 2C. A retention position may prevent tube 102 and tube retention device 100 from being easily removed from the body cavity. Removal may be prevented in the retention position because the largest dimension of the combined tube 102 and tube retention device 100 that is parallel to the body, shown as dimension A in FIG. 2B, is maximized. In this embodiment, dimension A may be larger than the surgical opening in the body 210 that allowed tube 102 and tube retention device 100 to enter the body cavity. Surgical opening 210 may be, for example, a stoma or other incision allowing access to the interior of a body. Removal may be prevented by the engagement of elongate segment 104 with the interior surface of the body cavity 208, as best shown in FIG. 2C.

Tube retention device 100 may reach a retention position when the largest dimension of elongate segment 104 is substantially perpendicular to tube 102. It may not be necessary for elongate segment 104 to be exactly parallel to tube 102 in a retention position, it may be sufficient for elongate segment 104 to be substantially perpendicular to tube 102, such that dimension A, as previously discussed, is larger than surgical opening 210 that allowed tube 102 and tube retention device 100 to enter the body cavity. Elongate segment 104 may be placed perpendicular to tube 102 by rotating elongate segment 104 about rotatable hinge 106. Elongate segment 104 may be rotated approximately ninety degrees from the insertion position to reach the retention position.

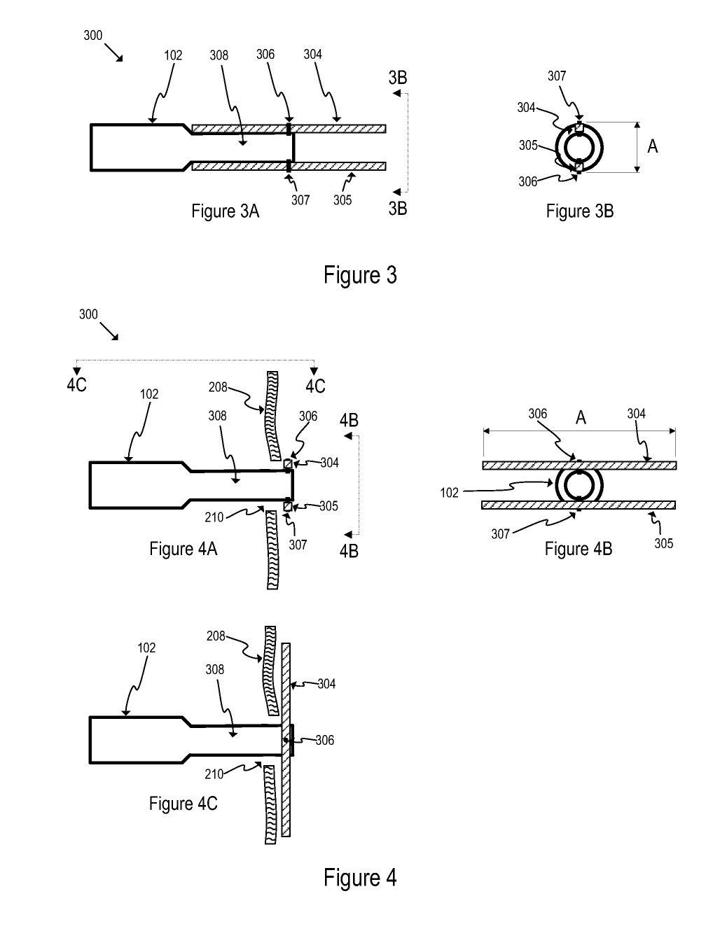

FIGS. 3A and 3B illustrate different viewpoints of another embodiment of the invention. FIG. 3A shows an elevation view of tube retention device 300 in an insertion position, while FIG. 3B shows a side view of tube retention device 300 in an insertion position. Similar elements will be referred to using the designations from FIGS. 1 and 2. In the embodiment in FIG. 3, tube retention device 300 may comprise two elongate segments 304, 305 attached to the distal end of tube 102 through two rotatable hinges 306, 307. Alternatively, a single rotatable hinge may attach both elongate segments 304, 305 to tube 102. Elongate segments 304, 305 may be located on opposing sides of tube 102, as shown in FIG. 3. Alternatively, elongate segments 304, 305 may be located anywhere on tube 102. In this embodiment, elongate segments 304, 305 are shown as elongated rectangular shapes, but they may be any shape, as previously discussed.

In this embodiment, the diameter of tube 102 may be reduced at distal end 308 to accommodate the addition of elongate segments 304, 305. Reducing the diameter of tube 102 may minimize the surgical opening in the body required to insert tube 102 and tube retention device 300 because the largest dimension of the combined tube 102 and tube retention device 300 that is parallel to the body, shown as dimension A in FIG. 3B, is minimized. The diameter of distal end 308 may be gradually reduced from the diameter of tube 102 near elongate segments 304, 305, as shown in FIG. 3A. Alternatively, the diameter of distal end 308 may be reduced anywhere proximal to elongate segments 304, 305 or may be reduced more or less gradually than is shown in FIG. 3A. The lumen of tube 102 may also be reduced at distal end 308, but still may be operable to accept a smaller diameter tube that extends from the exterior of the body into the body cavity.

FIGS. 3A and 3B show tube 102 and tube retention device 300 in an insertion position. As previously discussed, an insertion position may allow tube 102 and tube retention system 300 to enter the body cavity through the smallest possible surgical opening in the body because the largest dimension of the combined tube 102 and tube retention device 300 that is parallel to the body, shown as dimension A in FIG. 3B, is minimized. Tube retention device 300 may be in an insertion position when the largest dimension of elongate segments 304, 305 is substantially parallel to tube 102.

FIGS. 4A, 4B, and 4C illustrate different viewpoints of tube retention device 300 in a retention position. FIG. 4A shows an elevation view of tube retention device 300 in a retention position, FIG. 4B shows a side view of tube retention device 300 in a retention position, and FIG. 4C shows a top view of tube retention device 300 in a retention position. As previously discussed, a retention position may prevent tube 102 and tube retention device 300 from being easily removed from the body cavity. Removal may be prevented in the retention position because the largest dimension of the combined tube 102 and tube retention device 300 that is parallel to the body, shown as dimension A in FIG. 4B, is maximized. Removal may be prevented by the engagement of elongate segments 304, 305 with the interior surface of the body cavity 208, as best shown in FIG. 4C.

Tube retention device 300 may reach a retention position when the largest dimension of elongate segments 304, 305 are substantially perpendicular to tube 102. Tube retention device 300 may be shifted from an insertion position to a retention position by rotation of elongate segments 304, 305 from a position that is substantially parallel to tube 102 to a position that is substantially perpendicular to tube 102. Elongate segments 304, 305 may be rotated about rotatable hinges 306, 307.

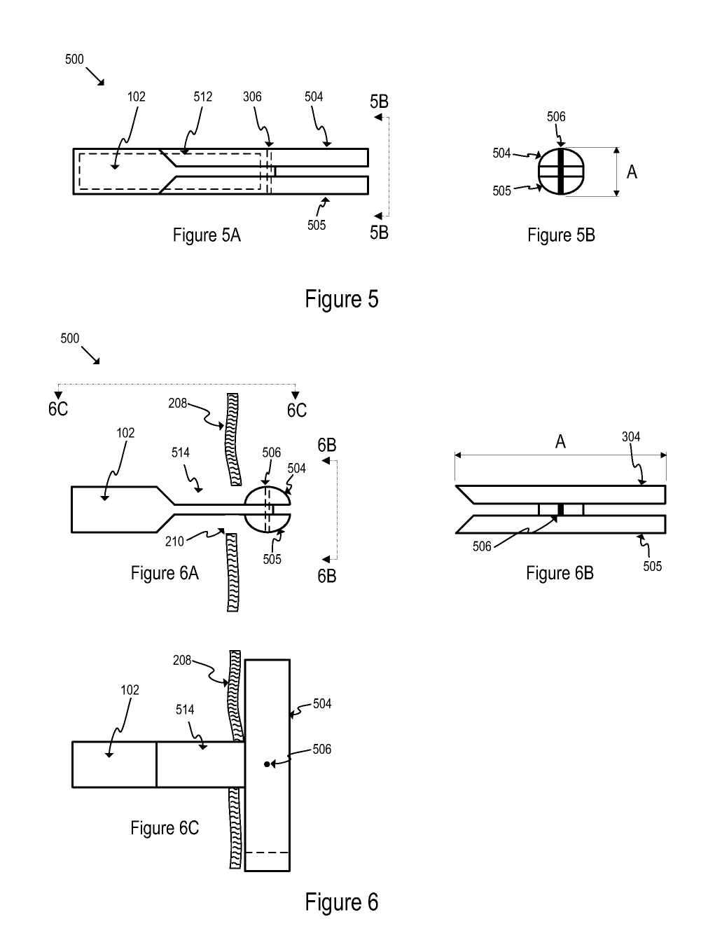

FIGS. 5A and 5B illustrate different viewpoints of another embodiment of the invention. FIG. 5A shows an elevation view of tube retention device 500 in an insertion position, while FIG. 5B shows a side view of tube retention device 500 in an insertion position. Similar elements will be referred to using the designations from FIGS. 1, 2, 3, and 4. In the embodiment in FIG. 5, tube retention device 500 may comprise two elongate segments 504, 505 attached to the distal end of tube 102 through a rotatable hinge 506. Rotatable hinge 506 is shown in hidden line in FIG. 5A because it may be contained within tube 102 and elongate segments 504, 505. Rotatable hinge 506 is more clearly shown in FIG. 5B because the ends of tube 102 and elongate segments 504, 505 are shown as open. However, the ends of tube 102 and elongate segments 504, 505 may be closed in other embodiments.

In this embodiment, elongate segments 504, 505 have an elongated hemispherical shape and are integrated into the distal end of tube 102, as shown in FIGS. 5A and 5B. Alternatively, elongate segments 504, 505 may be separate from tube 102 and may be designed to accommodate the shape of the distal end of tube 102. Alternatively, tube retention device 500 may include a tube element similar to tube 102, such that the tube element extends outside the body and attaches to another tube (not shown). Tube retention device 500 may include a lumen operable to accept a smaller diameter tube extending from the exterior of the body into the body cavity or operable to deliver products from the exterior of the body into the body cavity.

FIGS. 5A and 5B show tube 102 and tube retention device 500 in an insertion position. As previously discussed, an insertion position may allow tube 102 and tube retention system 500 to enter the body cavity through the smallest possible surgical opening in the body because the largest dimension of the combined tube 102 and tube retention device 500 that is parallel to the body, shown as dimension A in FIG. 5B, is minimized. Tube retention device 500 may be in an insertion position when the largest dimension of elongate segments 504, 505 is substantially parallel to tube 102.

Elongate segments 504, 505 may be held in an insertion position by a holding element 512. Holding element 512 is shown with tube retention device 500, but holding element 502 may be used with any embodiment of the invention. Holding element 512 is shown in hidden line in FIG. 5A because it may be contained within tube 102 and elongate segments 504, 505. Alternatively, holding element 512 may be located on the outside of tube 102 and elongate segments 504, 505. Holding element 512 may prevent elongate segments 504, 505 from rotating about rotatable hinge 506 by physically impeding their movement. Holding element 512 may be comprised of a material that is more rigid than elongate segments 504, 505. Holding element 512 may be comprised of any material that is suitable for insertion into a body, such as, for example, silicon, polyurethane, inert metal, or other biocompatible material. Holding element 512 may be removed from the proximal end of tube 102 after tube retention device 500 is located within a body cavity. After holding element 512 is removed, elongate segments 504, 505 may rotate about rotatable hinge 506 to shift from an insertion position to a retention position, as previously discussed.

Elongate segments 504, 505 may rotate about rotatable hinge 506 automatically due to a mechanism internal to tube retention device 500, such as, for example, a spring, piston, or tension in the hinge material. Alternatively, elongate segments 504, 505 may rotate about rotatable hinge 506 from an insertion position to a retention position with the assistance of an operator located at the proximal end of tube 102. Similarly, elongate segments 504, 505 may rotate about rotatable hinge 506 back to an insertion position with the assistance of an operator located at the proximal end of tube 102. The operator may assist the rotation of elongate segments 504, 505 by inserting an object into the lumen of tube 102 that applies force to elongate segments 504, 505. The force applied may create a moment arm used to rotate elongate segments 504, 505.

FIGS. 6A, 6B, and 6C illustrate different viewpoints of tube retention device 500 in a retention position. FIG. 6A shows an elevation view of tube retention device 500 in a retention position, FIG. 6B shows a side view of tube retention device 500 in a retention position, and FIG. 6C shows a top view of tube retention device 500 in a retention position. As previously discussed, a retention position may prevent tube 102 and tube retention device 500 from being easily removed from the body cavity. Removal may be prevented in the retention position because the largest dimension of the combined tube 102 and tube retention device 500 that is parallel to the body, shown as dimension A in FIG. 6B, is maximized. Removal may be prevented by the engagement of elongate segments 504, 505 with the interior surface of the body cavity 208, as best shown in FIG. 6C.

In this embodiment of the invention, rotation of elongate segments 504, 505 may expose an aperture 514 in tube 102 or tube retention device 500. Aperture 514 may exist on any side of tube 102 or tube retention device 500. Aperture 514 may allow a smaller diameter tube to be inserted into the body cavity through the lumen of tube 102 or tube retention device 500. Aperture 514 itself may be used to deliver products from the exterior of the body into the body cavity. The lumen of tube 102 or tube retention device 500 may extend past aperture 514 to the distal end. Alternatively, tube 102 or tube retention device 500 may include material to prevent an aperture being exposed when elongate segments 504, 505 rotate.

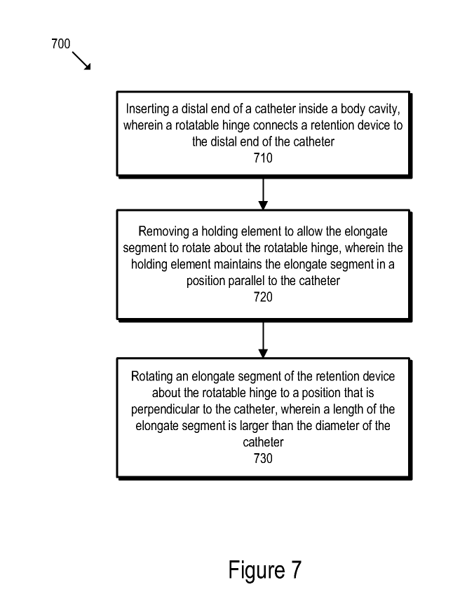

FIG. 7 illustrates method 700 for retaining a tube inside a body cavity according to another embodiment of the invention. The method begins with step 710 in which a distal end of a tube is inserted inside a body cavity. A rotatable hinge connects a retention device to the distal end of the tube. Step 720 involves removing a holding element from the retention device to allow the elongate segment to rotate about the rotatable hinge. The holding element may be used to maintain the elongate segment in a position parallel to the tube. Step 730 involves rotating an elongate segment of the retention device about the rotatable hinge to a position that is perpendicular to the tube. A length of the elongate segment may be larger than the diameter of the tube.

While various embodiments of the invention have been described, it will be apparent to those of ordinary skill in the art that many more embodiments and implementations are possible within the scope of the invention. Accordingly, the invention is not to be restricted except in light of the attached claims and their equivalents.

What is claimed is:

1. A device for retaining a tube inside a body cavity comprising:
an elongate segment attached to the distal end of a tube, wherein a length of the elongate segment is larger than the diameter of the tube and wherein the length of the elongate segment is operable to be substantially parallel to the tube in an insertion position and the length of the elongate segment is operable to be substantially perpendicular to the tube in a retention position; and
a rotatable hinge connecting the elongate segment to the distal end of the tube.

2. The device of claim 1, wherein the elongate segment is operable to rotate about the rotatable hinge from the insertion position to the retention position after the device reaches its destination in the body cavity.

3. The device of claim 1, wherein the rotatable hinge comprises a pin hinge, wherein a first end of the pin is rotatable connected to the elongate segment and a second end of the pin is rotatable connected to the tube.

4. The device of claim 1, wherein the elongate segment is operable to rotate about the rotatable hinge from the insertion position to the retention position without assistance from an operator after the device is contained in the body cavity.

5. The device of claim 1, wherein the elongate segment is operable to rotate about the rotatable hinge from the insertion position to the retention position with assistance from an operator after the device is contained in the body cavity.

6. The device of claim 1, further comprising a holding element operable to maintain the elongate segment in the insertion position until the holding element is removed.

7. The device of claim 1, wherein the rotatable hinge is located near the midpoint of the elongate segment.

8. The device of claim 1, wherein the elongate segment comprises two separate elongate segments.

9. The device of claim 8, wherein each of the two separate elongate segments are located on opposing sides of the device.

10. The device of claim 1, wherein the elongate segment is comprised of a resilient, biocompatible material.

11. The device of claim 1, wherein the elongate segment is comprised of silicone or polyurethane.

12. The device of claim 1, wherein the elongate segment is comprised of a material that is more rigid than the tube material.

13. The device of claim 1, further comprising a lumen operable to acceptable a smaller diameter tube that extends from outside the body to within the body cavity.

14. A catheter comprising:
a tube with a distal end and a proximal end, wherein the tube comprises a lumen connecting the distal end and the proximal end;
a retention device located at the distal end of the tube, wherein the retention device includes an elongate segment, wherein the length of the elongate segment is larger than the diameter of the tube and wherein the length of the elongate segment is operable to be parallel to the tube in an insertion position and the length of the elongate segment is operable to be perpendicular to the tube in a retention position; and
a rotatable hinge connecting the elongate segment to the distal end of the tube.

15. A method of retaining a tube inside a body cavity, the method comprising:
inserting a distal end of a tube inside a body cavity, wherein a rotatable hinge connects an elongate segment to the distal end of the tube;
rotating the elongate segment about the rotatable hinge to a position that is perpendicular to the tube, wherein a length of the elongate segment is larger than the diameter of the tube.

16. The method of claim 15, further comprising the step of removing a holding element in communication with the elongate segment to allow the elongate segment to rotate about the rotatable hinge, wherein the holding element maintains the elongate segment in a position parallel to the tube.
